(19) United States
(12) Patent Application Publication
(10) Pub. No.: US 2023/0181610 A1
(43) Pub. Date: Jun. 15, 2023

(54) Methods and Compositions for Treatment of Cystic Fibrosis Class I Mutations

(71) Applicant: Ann and Robert H. Lurie Children's Hospital of Chicago, Chicago, IL (US)

(72) Inventors: Hiroyuki Hirai, Chicago, IL (US); Youyang Zhao, Deerfield, IL (US)

(21) Appl. No.: 18/047,504

(22) Filed: Oct. 18, 2022

Related U.S. Application Data
(60) Provisional application No. 63/262,693, filed on Oct. 18, 2021.

(51) Int. Cl. A61K 31/7034; A61K 31/585; A61K 31/18; A61K 31/4439; A61K 31/404; A61K 31/47

(52) U.S. Cl. CPC A61K 31/7034; A61K 31/18; A61K 31/47; A61K 31/404; A61K 31/585; A61K 31/4439

(57) ABSTRACT

Disclosed are methods for treating cystic fibrosis characterized by a class I nonsense mutation in the cystic fibrosis transmembrane conductance regulator (CFTR) gene in a subject in need thereof. The method comprises administering to the subject a pharmaceutical composition comprising an effective amount of an inhibitor selected from the group consisting of a sodium glucose co-transporter (SGLT) inhibitor, a Na⁺/K⁺-ATPase inhibitor, an SGLT1/Na⁺/K⁺-ATPase dual inhibitor, and combinations thereof.

### CROSS REFERENCE TO RELATED APPLICATIONS

This application claims the benefit of priority of U.S. Provisional Pat. Application Ser. No. 63/262,693, filed Oct. 18, 2021, the contents of which is incorporated by reference in its entirety.

### REFERENCE TO AN ELECTRONIC SEQUENCE LISTING

The contents of the electronic sequence listing (702581.02247.xml; Size: 6,255 bytes; and Date of Creation: Oct. 17, 2022) is herein incorporated by reference in its entirety.

### BACKGROUND

Current cystic fibrosis transmembrane conductance regulator (CFTR) treatments include CFTR modulator therapies which treat cystic fibrosis characterized by class II, class III, class IV, and class VI mutations. However, there is no effective therapy for treating patients having cystic fibrosis characterized by class I CFTR nonsense mutations. As such, there exists a need for methods and compositions for treating cystic fibrosis characterized by class I CFTR nonsense mutations.

### SUMMARY

Disclosed are methods_for treating cystic fibrosis (CF) characterized by a class I nonsense mutation in the cystic fibrosis transmembrane conductance regulator (CFTR) gene_in a subject in need thereof.

In some embodiments, the disclosed methods may_comprise administering to the subject a pharmaceutical composition comprising an effective amount of an inhibitor selected from the group consisting of a sodium glucose co-transporter (SGLT) inhibitor, a Na⁺/K⁺-ATPase inhibitor, an SGLT⅟Na⁺/K⁺-ATPase dual inhibitor, and combinations thereof.

### BRIEF DESCRIPTION OF THE DRAWINGS

FIG. 1 shows generation of Proximal Lung Organoids from Cystic Fibrosis patient PSCs carrying Class I mutations. (A) Schematic representation of lung organoids differentiation protocol from CF patient PSCs (hiPSC). (B, C) Representative immunofluorescence staining for p63 and EpCAM in proximal Lung Organoids derived from PSCs with Class I mutations of either G542X/G542X (B) or W1282X/W1282X (C) on Day 22. Nuclei were counterstained with DAPI. Scale bars, 100 µm.

FIG. 2 shows_phlorizin promotes swelling of CF HLOs carrying CFTR class I mutation. (A) Time-lapse phase contrast images of forskolin (Fsk)-induced swelling of G542X/G542X HLOs treated with 100 µM Phlorizin (PHL), 10 µM Empagliflozin (Empa), or 20 µM Sotagliflozin (Sota). Scale bars represent 100 µm. (B) Quantification of organoid swelling of G542X/G542X HLOs treated with Phlorizin, Empagliflozin, or Sotagliflozin. (n=16). *** P<0.005.

FIG. 3 depicts SGLT1 and Na⁺/K⁺-ATPase dual inhibitor Phlorizin restores HLO swelling of Class I CFTR W1282X/W1282X mutation. (A) Phlorizin but neither Sotagliflozin nor ENaC inhibitor Amiloride restored the swelling function of HLOs with Class I CFTR W1282X/W1282X mutation. (n=15). (B) Quantification of organoid swelling of W1282X/W1282X HLOs treated with Phlorizin or TRIKAFTA (100 nM VX-770, 3 µM VX-445, and 3 µM VX-661). (n=10). (C) Quantification of organoid swelling of HLOs with Class II dF508/dF508 mutation treated with TRIKAFTA (100 nM VX-770 and 3 µM VX-445 and 3 µM VX-661). (n=18). * P<0.05; *** P<0.005.

FIG. 4 demonstrates_selective knockdown of SGLT1 with SLC5A1-specific shRNA partially restored the swelling function of Class I CFTR mutation HLOs. (A) Quantitative RT-PCR analysis demonstrating efficient knockdown of SLC5A1, i.e. SGLT1 by SLC5A1 shRNA in HLOs derived from PSCs carrying W1282X/W1282X mutation. (B) Quantification of organoid swelling of W1282X/W1282X mutation HLOs with either SLC5A1 shRNA knockdown of SGLT1 or control scrambled shRNA. *P<0.05.

FIG. 5 depicts shRNA-mediated knockdown of ATP1A1 encoding the a1 subunit of Na⁺/K⁺-ATPase partially restores the swelling function of HLOs carrying CFTR Class I mutation. (A) Quantitative RT-PCR analysis showing 50% knockdown of ATP1A1 expression in W1282X/W1282X HLOs. (B) Quantification of organoid swelling of W1282X/W1282X mutation HLOs. *P<0.05; **P<0.01.

FIG. 6 shows_Na⁺/K⁺-ATPase inhibitors partially restore HLO swelling of Class I CFTR mutations. (A) Quantification of organoid swelling of W1282X/W1282X HLOs treated with 100, 500 nM Ouabain or 20 µM Phlorizin. (n=9). (B) Quantification of organoid swelling of W1282X/W1282X HLOs treated with Chlorpropamide. (n=17). * P<0.05; **P < 0.01; *** P<0.005.

FIG. 7 shows that inhibition of both SGLT1 and Na⁺/K⁺-ATPase led to greater swelling effects of HLOs carrying Class I mutation. W1282X/W1282X HLOs were transfected with SLC5A1 shRNA to knockdown SGLT1 then treated with Forskolin plus Ouabain. 24 h later, the HLO sizes were quantified for the assessment of effects of dual inhibition of SGLT1 and Na⁺/K⁺-ATPase on restoration of swelling function compared to SGLT1 inhibition (i.e. shRNA-specific knockdown) alone.

### DETAILED DESCRIPTION

The present invention is described herein using several definitions, as set forth below and throughout the application.

Unless otherwise specified or indicated by context, the terms “a”, “an”, and “the” mean “one or more.” For example, “a sodium glucose co-transporter (SGLT) inhibitor” should be interpreted to mean “one or more sodium glucose co-transporter (SGLT) inhibitors.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Disclosed are methods for treating cystic fibrosis (CF) characterized by a class I nonsense mutation in the cystic fibrosis transmembrane conductance regulator (CFTR) gene in a subject in need thereof. The method comprises administering to the subject a pharmaceutical composition comprising an effective amount of an inhibitor selected from the group consisting of a sodium glucose co-transporter (SGLT) inhibitor, a Na⁺/K⁺-ATPase inhibitor, an SGLT1/Na⁺/K⁺-ATPase dual inhibitor, and combinations thereof.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disorder. As such, the methods disclosed herein encompass both therapeutic and prophylactic administration.

As used herein, the term “subject in need thereof” refers to a subject having or at risk for developing cystic fibrosis, including cystic fibrosis characterized by a class I nonsense mutation in the cystic fibrosis transmembrane conductance regulator (CFTR) gene. A “subject in need thereof” may include a human or non-human subject (e.g., a non-human mammal).

As used herein, the term “effective amount” refers to the amount or dose of the compound, upon single or multiple dose administration to the subject, which provides the desired effect in the subject under diagnosis or treatment. The disclosed methods may include administering an effective amount of the disclosed inhibitors (e.g., as present in a pharmaceutical composition) for treating cystic fibrosis (CF) characterized by a class I nonsense mutation in the cystic fibrosis transmembrane conductance regulator (CFTR) gene.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual patient; the particular inhibitor administered; the mode of administration; the bioavailability characteristics of the preparation administered; the dose regimen selected; the use of concomitant medication; and other relevant circumstances.

In some embodiments, a daily dose of the disclosed inhibitors may contain from about 0.01 mg/kg to about 100 mg/kg (such as from about 0.05 mg/kg to about 50 mg/kg and/or from about 0.1 mg/kg to about 25 mg/kg) of each inhibitor used in the present method of treatment. The dose may be administered under any suitable regimen (e.g., weekly, daily, twice daily).

As used herein, an “inhibitor” refers to a compound that inhibits the activity of a protein of interest, such as a SGLT or Na⁺/K⁺-ATPase protein. Suitably, the inhibitor may have a IC₅₀ of less than 1000 nM, less than 500 nM, less than 200 nM, less than 100 nM, less than 50 nM, less than 40 nM, less than 30 nM, less than 20 nM, less than 10 nM, or less than 5 nM for the protein of interest.

In some embodiments, the inhibitor used in the methods as described herein is an SGLT1/ Na⁺/K⁺-ATPase dual inhibitor. As used herein, the term “SGLT1/ Na⁺/K⁺-ATPase dual inhibitor” refers to an inhibitor that inhibits both SGLT1 protein and Na⁺/K⁺-ATPase.

In some embodiments, the SGLT1/ Na⁺/K⁺-ATPase dual inhibitor is phlorizin and analogs thereof.

As used herein the term “analog” refers to compounds having similar physical, chemical, biochemical, or pharmacological properties, which include structural analogs and/or functional analogs. The term “structural analog” or “chemical analog” refers to a compound having a structure similar to that of another compound, but differing with respect to one or more structural moieties (e.g. one or more atoms, functional groups, or substructures). A structural analog of a compound can theoretically be formed from that compound after one or more chemical reactions. The term “functional analog” may include compounds that are not necessarily structural analogs with a similar chemical structure. An example of pharmacological functional analogs are morphine, heroine, and fentanyl, which have the same mechanism of action, but fentanyl is structurally different from the other two.

The term “phlorizin” refers to a compound having a formula C₂₁H₂₄O₁₀ and an IUPAC name (1-(2,4-dihydroxy-6-{[(2S,3R,4S,5S,6R)-3,4,5-trihydroxy-6-(hydroxymethyl)oxan-2-yl]oxy}phenyl)-3-(4-hydroxyphenyl)propan-1-one). The chemical structure of phlorizin is shown below:

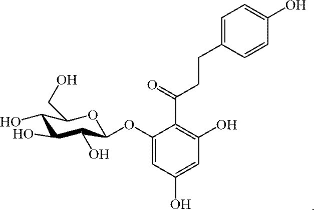

Phlorizin is a glucoside of phloretin, a dihydrochalcone, found primarily in unripe Malus (apple) root bark of apple. Phlorizin is an inhibitor of SGLT1 and SGLT2 because it competes with D-glucose for binding to the carrier. Phlorizin is also an inhibitor of Na⁺/K⁺-ATPase. Examples of phlorizin analogs include, but are not limited to, empagliflozin, canagliflozin and dapagliflozin.

In some embodiments, the inhibitor used in the methods as described herein is an SGLT inhibitor. In some embodiments, the SGLT inhibitor used in the methods as described herein is a non-selective SGLT inhibitor. In other embodiments, the SGLT inhibitor is a SGLT1-selective inhibitor.

As used herein, the term “non-selective SGLT inhibitor” refers to an inhibitor that inhibits both SGLT1 and SGLT2 proteins. The selectivity of an inhibitor for SGLT1 or SGLT2 protein may be measured by the value Ki, which is the dissociation constant describing the binding affinity between the inhibitor and the enzyme, or the IC₅₀. In some embodiments, a non-selective SGLT inhibitor has an approximately equal (50/50) selectivity for SGLT1 and SGLT2 proteins.

As used herein, the term “SGLT1-selective inhibitor” refers to an inhibitor that has a greater inhibitory affect against SGLT1 than SGLT2. In some embodiments, the SGLT1-selective inhibitor may have an IC₅₀ for SGLT2 that is at least 5, 10, 20, 50, or 100 times greater than for SGLT1.

In some embodiments, the SGLT inhibitor is selected from the group consisting of sotagliflozin, phloretin, licogliflozin, SGLT inhibitor 1, mizagliflozin, KGA-2727, SGL5213, LX2761, T-1095, analogs thereof, and any combination thereof.

The term “sotagliflozin” refers to a compound having a chemical formula C21H25C1O5S and an IUPAC name (2S,3R,4R,5S,6R)-2-[4-chloro-3-[(4-ethoxyphenyl)methyl]phenyl]-6-methylsulfanyloxane-3,4,5-triol. The structure of sotagliflozin is shown below:

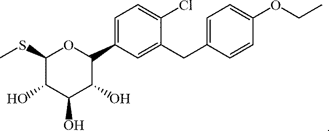

Sotagliflozin is an orally bioavailable inhibitor of the sodium-glucose co-transporter subtype 1 (SGLT1) and 2 (SGLT2), with potential antihyperglycemic activity. Upon oral administration, sotagliflozin binds to and blocks both SGLT1 in the gastrointestinal (GI) tract and SGLT2 in the kidneys, thereby suppressing the absorption of glucose from the GI tract and the reabsorption of glucose by the proximal tubule into the bloodstream, respectively.

The term “phloretin” refers to a compound having a chemical formula C₁₅H₁₄O₅ and an IUPAC name 3-(4-hydroxyphenyl)-1-(2,4,6-trihydroxyphenyl)propan-1-one. The structure of phloretin is shown below:

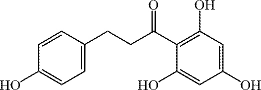 [SMILES: O=C(CCc1ccc(O)cc1)c1c(O)cc(O)cc1O]

Phloretin is a flavonoid extracted from Malus pumila Mill. and has antiinflammatory activities. Phloretin is a specific, competitive and orally active inhibitor of sodium/glucose cotransporter SGLT½.

The term “licogliflozin” refers to a compound having a chemical formula C₂₃H₂₈O₇ and an IUPAC name (2S,3R,4R,5S,6R)-2-[3-(2,3-dihydro-1,4-benzodioxin-6-ylmethyl)-4-ethylphenyl]-6-(hydroxymethyl)oxane-3,4,5-triol. The structure of licogliflozin is shown below:

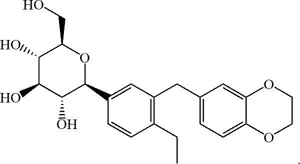

Licogliflozin (LIK066) is a non-anti-fibrotic treatment agent for non-alcoholic steatohepatitis (NASH). Licogliflozin is SGLT½ inhibitor.

The term “SGLT inhibitor 1” refers to a compound having a chemical formula C₂₄H₂₇FO₈ and a structure as shown below:

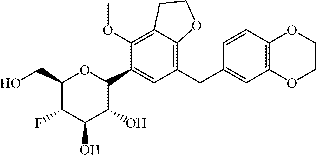

SGLT inhibitor 1 is a potent dual inhibitor of sodium glucose co-transporter proteins (SGLTs), inhibits hSGLT1 and hSGLT2 with IC₅₀s of 43 nM and 9 nM, respectively.

The term “mizagliflozin” refers to a compound having a chemical formula C₂₈H₄₄N₄O₈ and an IUPAC name 2,2-dimethyl-3-[3-[3-methyl-4-[[5-propan-2-yl-3-[(2S,3R,4S,5S,6R)-3,4,5-trihydroxy-6-(hydroxymethyl)oxan-2-yl]oxy-1H-pyrazol-4-yl]methyl]phenoxy]propylamino]propanamide. The structure of mizagliflozin is shown below:

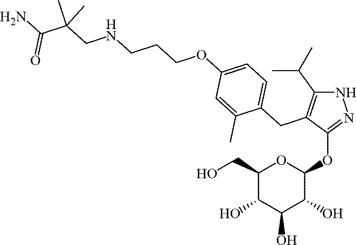 [SMILES: Cc1cc(OCCCNCC(C)(C)C(N)=O)ccc1Cc1c(O[C@@H]2O[C@H](CO)[C@@H](O)[C@H](O)[C@H]2O)n[nH]c1C(C)C]

Mizagliflozin is a potent, orally active and selective SGLT1 inhibitor, with a Kᵢ of 27 nM for human SGLT1. Mizagliflozin displays 303-fold selectivity over SGLT2. Mizagliflozin is used as an antidiabetic drug that can modify postprandial blood glucose excursion. Mizagliflozin also exhibits potential in the amelioration of chronic constipation.

The term “KGA-2727” refers to a compound having a chemical formula C₂₆H₄₀N₄O₈ and a chemical name 3-((3-(4-((5-isopropyl-3-(((2S,3R,4S,5S,6R)-3,4,5-trihydroxy-6-(hydroxymethyl)tetrahydro-2H-pyran-2-yl)oxy)-1H-pyrazol-4-yl)methyl)-3-methylphenoxy)propyl)amino)propanamide. The structure of KGA-2727 is shown below:

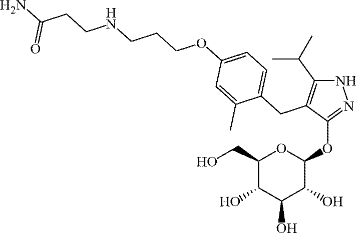 [SMILES: Cc1cc(OCCCNCCC(N)=O)ccc1Cc1c(O[C@@H]2O[C@H](CO)[C@@H](O)[C@H](O)[C@H]2O)n[nH]c1C(C)C]

KGA-2727 is a selective, high-affinity and orally active SGLT1 inhibitor with Kᵢs of 97.4 nM for human SGLT1.

The term “SGL5213” refers to a compound having a chemical formula C₂₆H₄₀N₄O₈ and an IUPAC name (E)-N-(1-((2-(dimethylamino)ethyl)amino)-2-methyl-1-oxopropan-2-yl)-4-(4-(2-isopropyl-4-methoxy-5-((2S,3R,4R,5S,6R)-3,4,5-trihydroxy-6-(hydroxymethyl)tetrahydro-2H-pyran-2-yl)benzyl)phenyl)-2,2-dimethylbut-3-enamide. The structure of SGL5213 is shown below:

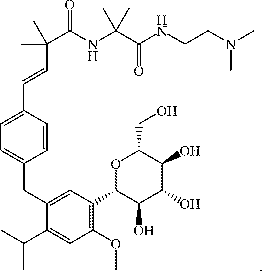

SGL5213 is a potent, orally active and low-absorbable sodium-dependent glucose cotransporter 1 (SGLT1) inhibitor, with IC₅₀ values of 29 nM and 20 nM for hSGLT1 and hSGLT2, respectively.

The term “LX2761” refers to a compound having a chemical formula C₃₂H₄₇N₃O₆S and an IUPAC name N-(1-((2-(Dimethylamino)ethyl)amino)-2-methyl-1-oxopropan-2-yl)-4-(4-(2-methyl-5-((2S,3R,4R,5S,6R)-3,4,5-trihydroxy-6-(methylthio)tetrahydro-2H-pyran-2-yl)benzyl)phenyl)-butanamide. The structure of LX2761 is shown below:

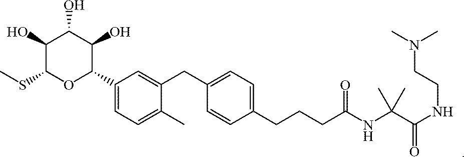

LX2761 is a potent inhibitor against SGLT1 and SGLT2 in vitro with IC₅₀s of 2.2 nM and 2.7 nM for hSGLT1 and hSGLT2, but displays specific SGLT1 inhibition in the gastrointestinal tract.

The term “T-1095” refers to a compound having a chemical formula C₂₆H₂₈O₁₁, and a chemical name ((2A,35,45,5A,65)-6-(2-(3-(benzofuran-5-yl)propanoyl)-3-hydroxy-5-methylphenoxy)-3,4,5-trihydroxytetrahydro-2H-pyran-2-yl)methyl methyl carbonate. The structure of T-1095 is shown below:

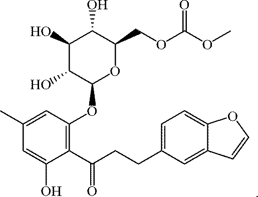

T-1095 is a selective and orally active Na⁺-glucose cotransporter (SGLT) inhibitor with IC₅₀s of 22.8 µM and 2.3 µM for human SGLT1 and SGLT2, respectively.

In some embodiments, the inhibitor used in the methods described herein is a Na⁺/K⁺-ATPase inhibitor. In some embodiments, the Na⁺/K⁺-ATPase inhibitor is selected from the group consisting of ouabain, bufalin, istaroxime, biacetyl monoxime, rostafuroxin, gitoxin, oleandrin, deslanoside, chloropropamide, periplocin, analogs thereof, and any combination thereof.

The term “ouabain” refers to a compound having a_chemical formula C₂₉H₄₄O₁₂ and a chemical name 3-[(1R,3S,5S,8R,9S,10R,11R,13R,14S,17R)-1,5,11,14-tetrahydroxy-10-(hydroxymethyl)-13-methyl-3-[(2R,3R,4R,5R,6S)-3,4,5-trihydroxy-6-methyloxan-2-yl]oxy-2,3,4,6,7,8,9,11,12,15,16,17-dodecahydro-1H-cyclopenta[a]phenanthren-17-yl]-2N-furan-5-one. The structure of ouabain is shown below:

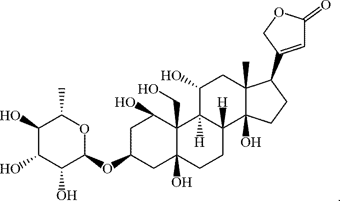

Ouabain is a cardiac glycoside that acts by inhibiting the Na+/K+-ATPase sodium-potassium ion pump (but it is not selective). Intravenous ouabain has a long history in the treatment of heart failure, and some continue to advocate its use intravenously and orally in angina pectoris and myocardial infarction. The trade name of ouabain is Strodival.

The term “bufalin” refers to a compound having a chemical formula C₂₄H₃₄O₄ and a chemical name 5-[(3S,5R,8R,9S,10S,13R,14S,17R)-3,14-dihydroxy-10,13-dimethyl-1,2,3,4,5,6,7,8,9,11,12,15,16,17-tetradecahydrocyclopenta[a]phenanthren-17-yl]pyran-2-one. The structure of bufalin is shown below:

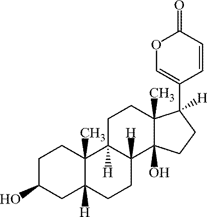

The term “istaroxime” refers to a compound having a chemical formula C₂₁H₃₂N₂O₃ and a chemical name (3E,5S,8R,9S,10R,13S,14S)-3-(2-aminoethoxyimino)-10,13-dimethyl-1,2,4,5,7,8,9,11,12,14,15,16-dodecahydrocyclopenta[a]phenanthrene-6,17-dione. The structure of istaroxime is shown below:

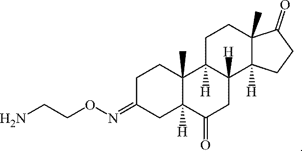

The term “biacetyl monoxime” refers to a compound having a chemical formula C₄H₇NO₂. The structure of biacetyl monoxime is shown below:

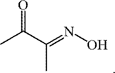

The term “rostafuroxin” refers to a compound having a chemical formula C₂₃H₃₄O₄ and a chemical name (3β,5β,14β)-21,23-epoxy-24-norchola-20,22-diene-3,14,17-triol. The structure of rostafuroxin is shown below:

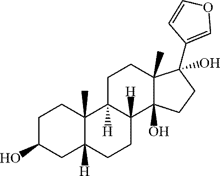

The term “gitoxin” refers to a compound having a chemical formula C₄₁H₆₄O₁₄ and an IUPAC name 3-[(3S,5R,8R,9S,10S,13R,14S,16S,17R)-3-[(2R,4S,5S,6R)-5-[(2S,4S,5S,6R)-5-[(2S,4S,5S,6R)-4,5-dihydroxy-6-methyloxan-2-yl]oxy-4-hydroxy-6-methyloxan-2-yl]oxy-4-hydroxy-6-methyloxan-2-yl]oxy-14,16-dihydroxy-10,13-dimethyl-1,2,3,4,5,6,7,8,9,11,12,15,16,17-tetradecahydrocydopenta[a]phenanthren-17-yl]-2H-furan-5-one. The structure of gitoxin is shown below:

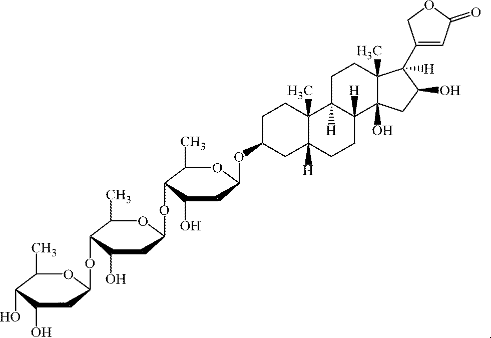

The term “oleandrin” refers to a compound having a chemical formula C₃₂H₄₈O₉ and an IUPAC name [(3S,5R,8R,,10S,13R,14S,16S,17R)-14-hydroxy-3-[(2R,4S,5S,6S)-5-hydroxy-4-methoxy-6-methyloxan-2-yl]oxy-10,13-dimethyl-17-(5-oxo-2H-furan-3-yl)-1,2,3,4,5,6,7,8,9,11,12,15,16,17-tetradecahydrocyclopenta[a]phenanthren-16-yl] acetate. The structure of gitoxin is shown below:

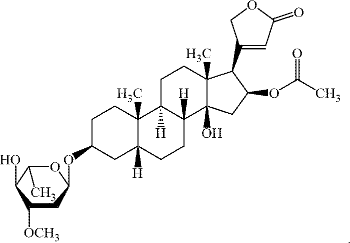

The term “deslanoside” refers to a compound having a chemical formula C₄₇H₇₄O1₉ and an IUPAC name 3-[(3S,5R,8R,9S,10S,12R,13S,14S,17R)-12,14-dihydroxy-3-[(2R,4S,5S,6R)-4-hydroxy-5-[(2S,4S,5S,6R)-4-hydroxy-5-[(2S,4S,5S,6R)-4-hydroxy-6-methyl-5-[(2S,3R,4S,5S,6R)-3,4,5-trihydroxy-6-(hydroxymethyl)oxan-2-yl]oxyoxan-2-yl]oxy-6-methyloxan-2-yl]oxy-6-methyloxan-2-yl]oxy-10,13-dimethyl-1,2,3,4,5,6,7,8,9,11,12,15,16,17-tetradecahydrocyclopenta[a]phenanthren-17-yl]-2H-furan-5-one. The structure of deslanoside is shown below:

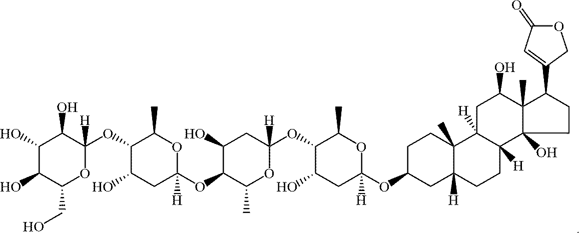

The term “chloropropamide” refers to a compound having a chemical formula C₁₀H₁₃ClN₂O₃S and a chemical name 4-chloro-N-(propylcarbamoyl)benzenesulfonamide. The structure of chloropropamide is shown below:

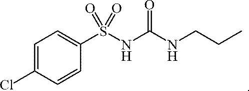

Chlorpropamide inhibits Na⁺/K⁺-ATPase. Chlorpropamide is also a drug in the sulfonylurea class used to treat non-insulin-dependent diabetes mellitus type 2. It is a long-acting first-generation sulfonylurea which causes relatively long episodes of hypoglycemia; gliclazide or tolbutamide are shorter-acting sulfonylureas. Trade names include Abemide and diabinese.

The term “periplocin” refers to a compound having a formula C₃₆H₅₆O₁₃ and a chemical name 3-[(3S,5S,8R,9S,10R,13R,14S,17R)-5,14-dihydroxy-3-[(2R,4S,5R,6R)-4-methoxy-6-methyl-5-[(2S,3R,4S,5S,6R)-3,4,5-trihydroxy-6-(hydroxymethyl)oxan-2-yl]oxyoxan-2-yl]oxy-10,13-dimethyl-2,3,4,6,7,8,9,11,12,15,16,17-dodecahydro-1H-cyclopenta[a]phenanthren-17-yl]-277-furan-5-one. The structure of marinobufogenin is shown below:

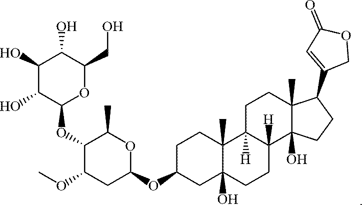

In some embodiments,_the Na⁺/K⁺-ATPase inhibitor is ouabain or analogs thereof.

In some embodiments, the Na⁺/K⁺-ATPase inhibitor is chlorpropamide or analogs thereof.

In some embodiments, the methods or pharmaceutical compositions as described herein may employ combination therapies, which means that a subject is administered at least two different active agents. For example, the active agents may be combined and administered in a single dosage form, may be administered as separate dosage forms at the same time, or may be administered as separate dosage forms that are administered alternately or sequentially on the same or separate days. In some embodiments, the active agents are combined and administered in a single dosage form. In some embodiments, the active agents are administered in separate dosage forms. In some embodiments, such combination therapies utilize lower dosages of the conventional therapeutics, thus avoiding possible toxicity and adverse side effects incurred when those agents are used as monotherapies.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic.

The agents, whether administered alone or in combination, may be administered multiple times, and if administered as a combination, may be administered simultaneously or not, and on the same schedule or not. By way of example, a therapeutic composition may be administered one or more times per day, one or more times per week, one or more times per month, or as often as a doctor prescribes.

In some embodiments, when administered in combination, the two or more agents can have a synergistic effect. As used herein, the terms “synergy” or “synergistic” refers to circumstances under which the biological activity of a combination of an agent and at least one additional therapeutic agent is greater than the sum of the biological activities of the respective agents when administered individually.

In some embodiments, the methods or pharmaceutical composition comprises the use of a SGLT inhibitor and a Na⁺/K⁺-ATPase inhibitor.

In some embodiments, the methods or pharmaceutical composition comprises the use of a SGLT1-selective inhibitor and a Na⁺/K⁺-ATPase inhibitor.

In some embodiments, the methods or pharmaceutical composition comprises the use of a non-selective SGLT inhibitor and a Na⁺/K⁺-ATPase inhibitor.

In some such embodiments, the methods or pharmaceutical compositions further comprise the use of a therapeutic agent selected from the group consisting of a CFTR modulator, a CFTR amplifier, and combinations thereof.

As used herein, the term “CFTR modulator” refers to CFTR potentiators and/or CFTR correctors. Examples of CFTR potentiators include, but are not limited to, Ivacaftor (VX-770), CTP-656, NVS-QBW251, FD1860293, and N-(3-carbamoyl-5,5,7,7-tetramethyl-4,7-dihydro-5H-thieno[2,3-c]pyran-2-yl)-1H-pyrazole-5-carboxamide.

Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, and WO2013038390; and U.S. Applications 14/271,080 and 14/451,619.

Examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661), VX-983, GLPG2222, GLPG2665, GLPG2737, VX-152, VX-440, FDL169, FDL304, FD2052160, and FD2035659. Examples of correctors are also disclosed in US20160095858A1, and U.S. Applications 14/925,649 and 14/926,727.

As used herein, the term “CFTR amplifier” refers to therapeutic agents that enhance the effect of known CFTR modulators, such as potentiators and correctors. An example of a CFTR amplifier is PTI130. Examples of amplifiers are also disclosed in publications WO2015138909 and WO2015138934.

In some embodiments, the therapeutic agent included in the methods as described herein is a CFTR modulator selected from the group consisting of Trikafta, Symdeko, Kalydeco, Orkambi, and combinations thereof.

The term “Trikafta” or “Kaftrio,” is the brand name for the combination of elexacaftor, tezacaftor, and ivacaftor. Trikafta is a prescription medicine used for the treatment of cystic fibrosis (CF) in patients who have at least one copy of the F508del mutation in the cystic fibrosis transmembrane conductance regulator (CFTR) gene or another mutation that is responsive to treatment with Trikafta.

The term “Symdeko” is the brand name for the combination of tezacaftor and ivacaftor. Symdeko is used for treatment of patients with cystic fibrosis (CF) who are homozygous for the F508del mutation or who have at least one mutation in the cystic fibrosis transmembrane conductance regulator (CFTR) gene that is responsive to tezacaftor/ivacaftor based on in vitro data and/or clinical evidence.

The term “Kalydeco” is the brand name for ivacaftor, which has a chemical formula C₂₄H₂₈N₂O₃ and a chemical name N-(2,4-ditert-butyl-5-hydroxyphenyl)-4-oxo-1H-quinoline-3-carboxamide.

The term “Orkambi” is the brand name for the combination of lumacaftor and ivacaftor that is used to treat people with cystic fibrosis who have two copies of the F508del mutation.

In some embodiments, the therapeutic agent included in the composition is a CFTR modulator that is Trikafta.

In some embodiments, the pharmaceutical composition further comprises one or more pharmaceutically acceptable carrier, excipient, or diluent. As one skilled in the art will also appreciate, the disclosed pharmaceutical compositions can be prepared with materials (e.g., actives excipients, carriers, and diluents etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The inhibitors utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. Alternatively, the inhibitors utilized in the methods disclosed herein may be formulated as a pharmaceutical composition in liquid form (e.g., an injectable liquid or gel)

The inhibitors utilized in the methods disclosed herein also may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents for the pharmaceutical compositions may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

In some embodiments, the pharmaceutical compositions as described herein can be administered in combination with adjuvants that enhance stability of the agents, facilitate administration of pharmaceutical compositions containing them in certain embodiments, provide increased dissolution or dispersion, increase activity, provide adjuvant therapy, and the like, including other active ingredients.

Pharmaceutical compositions comprising the compounds may be adapted for administration by any appropriate route, for example by the oral (including buccal or sublingual), nasal, inhalation, parenteral (including subcutaneous, intramuscular, intravenous or intradermal) route, or direct injection or administration to a tumor. Such formulations may be prepared by any method known in the art of pharmacy, for example by bringing into association the active ingredient with the carrier(s) or excipient(s).

Pharmaceutical compositions adapted for oral administration may be presented as discrete units such as capsules, pills, tablets, powders, granules, dragees, liquids, gels, syrups; slurries, solutions, or suspensions in aqueous or non-aqueous liquids; edible foams or whips; or oil-in-water liquid emulsions or water-in-oil liquid emulsions.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators. For nasal or inhalation delivery, the compositions of the disclosure also may be formulated by methods known to those of skill in the art, and may include, for example, sprays, inhalers, vapors; solubilizing, diluting, or dispersing substances, such as saline, preservatives, such as benzyl alcohol; absorption promoters; and fluorocarbons may be included.

Pharmaceutical compositions adapted for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

In some embodiments, the composition comprises phlorizin and Trikafta.

In some embodiments, the composition comprises ouabain and Trikafta. In some embodiments, the composition comprises chlorpropamide and Trikafta.

In some embodiments, the class I nonsense mutation is selected from the group consisting of a G542X mutation, a W1282X mutation, and combinations thereof.

### EXAMPLES

The following Examples are illustrative and should not be interpreted to limit the scope of the claimed subject matter.

By employing the isogenic human proximal lung organoids (HLOs) from gene-edited patient-derived pluripotent stem cells (PSCs) carrying different CFTR mutations, it was discovered that a sodium/glucose cotransporter ½ (SGLT½) and Na⁺/K⁺-ATPase dual inhibitor, such as Phlorizin (PHL), unexpectedly promoted forskolin-stimulated HLO swelling of CF HLOs derived from PSCs harboring class I CFTR mutations G542X/G542X, or W1282X/W1282X. shRNA-mediated knockdown of either SLC5A1 (encoding SGLT1) or ATP1A1 (encoding the a1 subunit of Na⁺/K⁺-ATPase) also promoted forskolin-stimulated swelling of CF HLOs with class I mutations. The Example also shows that Na⁺/K⁺-ATPase inhibitors, such as Ouabain and Chlorpropamide, also restored the defective function of swelling of CF HLO with Class I mutations. Together, these results demonstrate that SGLT1 and Na⁺/K⁺-ATPase are therapeutic targets for CF with Class I mutations. Thus, SGLT1 inhibitors and Na⁺/K⁺-ATPase inhibitors and more importantly SGLT1 and Na⁺/K⁺-ATPase dual inhibitors, such as PHL allow for treatment of Cystic Fibrosis with Class I mutations.

### Introduction

Mutations in the cystic fibrosis transmembrane conductance regulator (CFTR) gene often lead to cystic fibrosis (CF), a lethal autosomal-recessive inherited disease (1). In October 2019, the U.S. Food and Drug Administration (FDA) approved Trikafta, which provides benefits to more than 90% of CF patients aged 12 and older who have at least one copy of the most common CF mutation, F508del including Class II, III, IV and VI mutations (2). Although the community celebrated this milestone achievement 30 years after the discovery the CFTR gene (3), the consensus remains that this marks a new start of our efforts in developing new and effective therapeutics for CF, as the disease is not cured yet, especially because Trikafta does not benefit those with two nonsense mutations or other hard-to treat-mutations such as Class I mutations.

Among animal and cellular models for the study of CF and CFTR, organoids, three dimensional (3D) in vitro cell cultures that can be derived using either primary cells or pluripotent stem cells (PSCs) have emerged as a model of choice. For example, CF intestine organoids recapitulating essential features of the in vivo tissue architecture were first derived using patient primary intestine epithelial cells (4). More recently, in 2017, McCauley et al. (5) reported that Wnt signaling regulates lung differentiation of PSCs and that low-Wnt conditions allowed derivation of human proximal lung organoids (HLOs) from purified PSC-derived lung epithelial cells. These intestine and lung organoids, when subjected to a forskolin (Fsk)-stimulated swelling assay, respond in a mutation-dependent manner: normal CFTR wild-type (WT) organoids swell rapidly, whereas CF organoids show minimal expansion of size. Furthermore, the extent of swelling corresponds quantitatively with Fsk-induced anion currents (4). The swelling assay hence represents a simple and robust method to measure CFTR function in the organoids.

Sodium-dependent glucose cotransporters 1 and 2 (SGLT½) belong to the family of glucose transporters, encoded by SLC5A1 and SLC5A2 (6) respectively. SGLT2 is almost exclusively expressed in the apical membrane of the renal proximal convoluted tubule cells, a site that is minimally affected in CF. SGLT1 is also expressed in the kidneys. Unlike SGLT2, SGLT1 is additionally expressed in many other tissues, including CF-relevant ones such as the lungs and the intestine.

In the present work, we derived HLOs using CFTR isogenic PSCs with Class I mutations and found that SGLT1 and Na⁺/K⁺-ATPase are therapeutic targets for CF with Class I mutations. The results provide evidence that SGLT1 inhibitors, Na⁺/K⁺-ATPase inhibitors, and SGLT1 and Na⁺/K⁺-ATPase dual inhibitors, such as PHL and its analogs, are the long-sought therapies for Cystic Fibrosis with Class I mutations.

### Results and Discussion

### Derivation of HLOs Using PSCs Carrying CFTR Class I Mutations

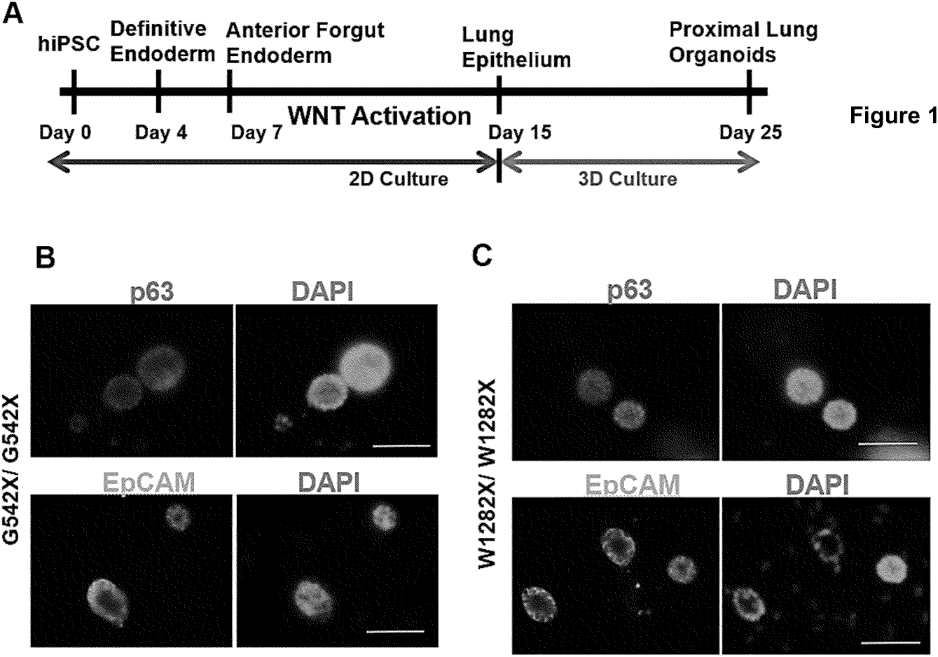

HLOs from PSCs carrying the homozygous CFTR nonsense mutations G542X or W1282X (Cystic Fibrosis Foundation), which are Class I mutations (7) were established in vitro following a 22-day regime (FIG. 1A) as previously described (5, 8, 9) (FIG. 1A). Both G542X/G542X and W1282X/W1282X HLOs, were positive for p63, and EpCAM consistent with their fate into proximate lung lineage cells (FIGS. 1B and 1C), demonstrating the derivation of HLOs carrying CFTR class I mutations.

### SGLT½ Non-Selective Inhibitor Phlorizin But Neither SGLT2-Slective Inhibitor Nor Trikafta Restores HLO Swelling of Class I CFTR Mutations

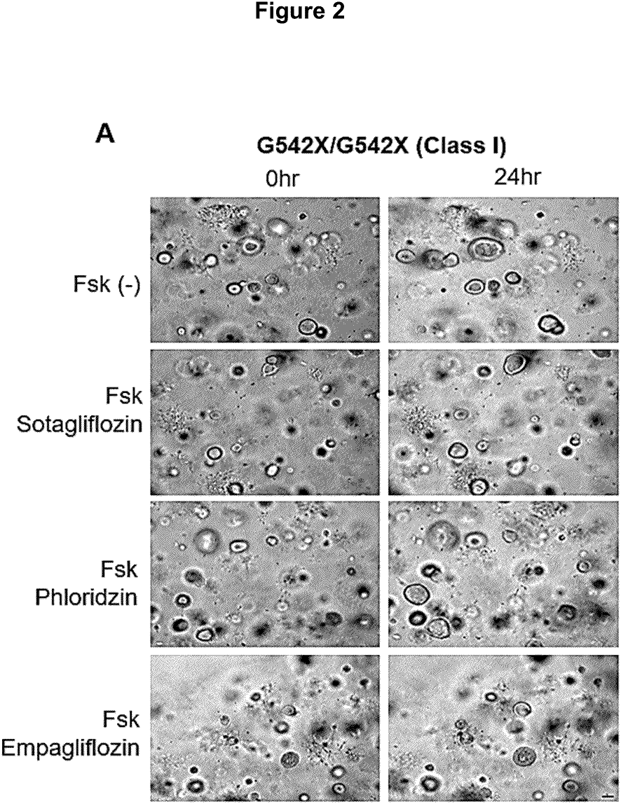

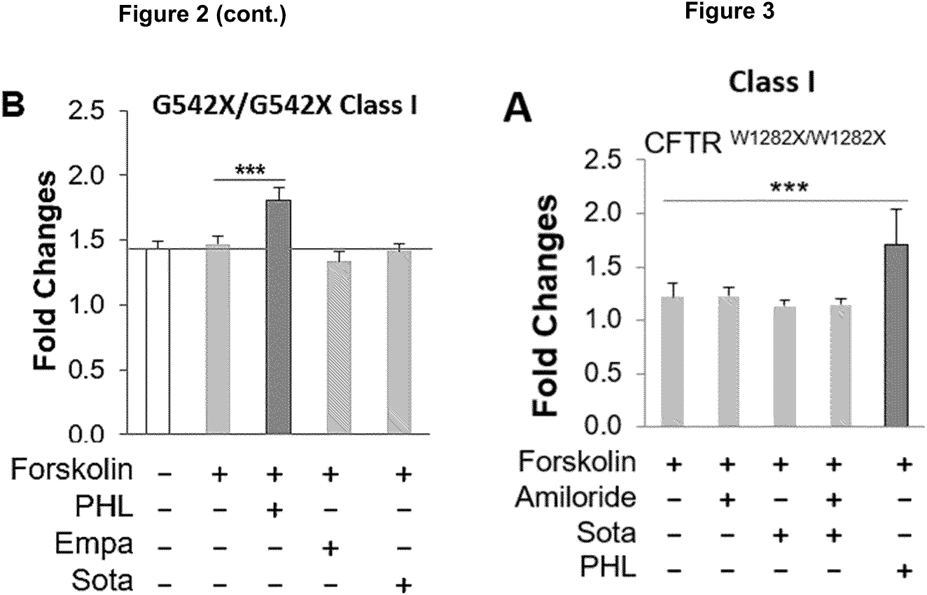

The G542X/G542XHLOs were subjected to fsk-stimulated swelling assay. As shown in FIG. 2, Phlorizin (1:1 selectivity between SGLT1 and SGLT2) but not Empagliflozin (2,500-fold higher selectivity for SGLT2 over SGLT1) treatment markedly increased the size of the HLOs. Next, we also assessed the effects of Sotagliflozin which has 20-fold higher selectivity for SGLT2 over that of SGLT1. Surprisingly, Sotagliflozin had no effect on G542X/G542X HLO swelling similar to Empagliflozin in contrast to Phlorizin (FIG. 2B).

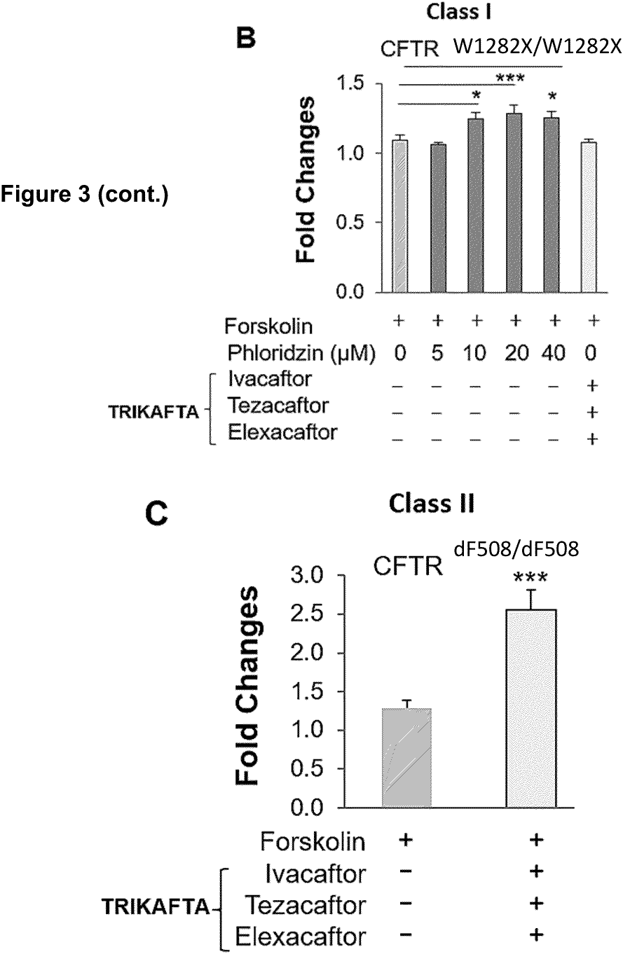

We next assessed the swelling effects on HLOs with different Class I mutation, W1282X/W1282X. Again, only Phlorizin but not Sotagliflozin markedly increased the size of swelling (FIG. 3A). Amiloride, an epithelium sodium channel (ENaC) inhibitor also had no stimulating swelling effects in this HLOs with Class I mutation (FIG. 3A). Then we also addressed the swelling effects of Trikafta on W1282X/W1282X HLOs. Trikafta could not promote fsk-stimulated W1282X/W1282X HLO swelling (FIG. 3B). However, as expected, it could markedly promote fsk-stimulated swelling of HLOs with dF508/dF508 mutation, a classical class II mutation (FIG. 3C), which is consistent with the clinical observation that Trikafta is beneficial to CF patients with Class II, III, IV and VI mutations but not Class I mutations.

### Selective Inhibition of SGLT1 by shRNA-Mediated Knockdown Promotes Swelling of HLOs With Class I Mutation

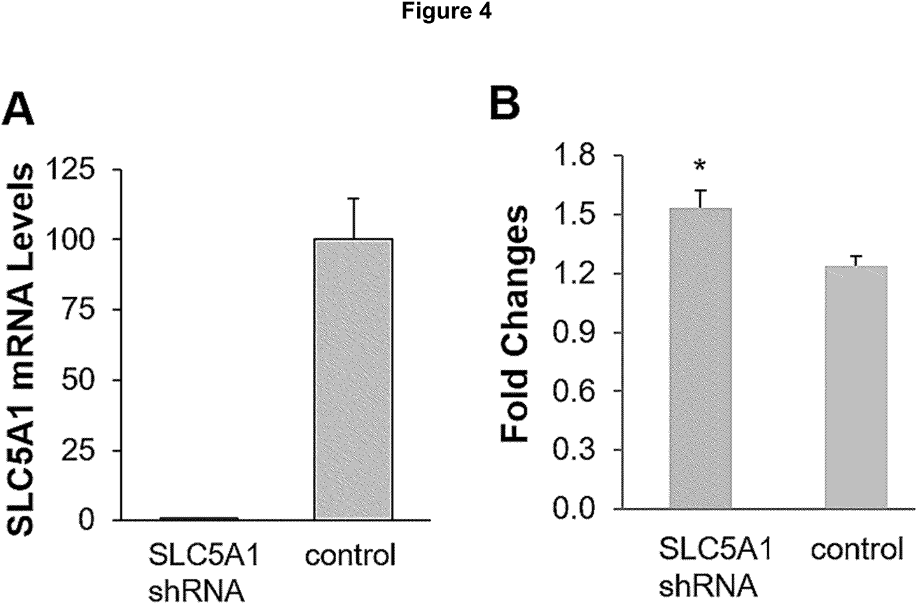

Different from Sotagliflozin and Empagliflozin, Phlorizin has no selectivity between SGLT1 and SGLT2. To determine if SGLT1 is drug target for CF Class I mutations, we employed SGLT 1-specific shRNA to knockdown SGLT1 in HLOs with Class I mutation (W1282X/W1282X) (FIG. 4A). Fsk-stimulated swelling assay showed significant restoration of swelling function of SGLT1-deficient HLOs (24% increase) (FIG. 4B), demonstrating SGLT1 is therapeutic drug target for CF patients with Class I mutations.

### Na⁺/K⁺-ATPase Inhibitors Restore HLO Swelling of Class I CFTR Mutations

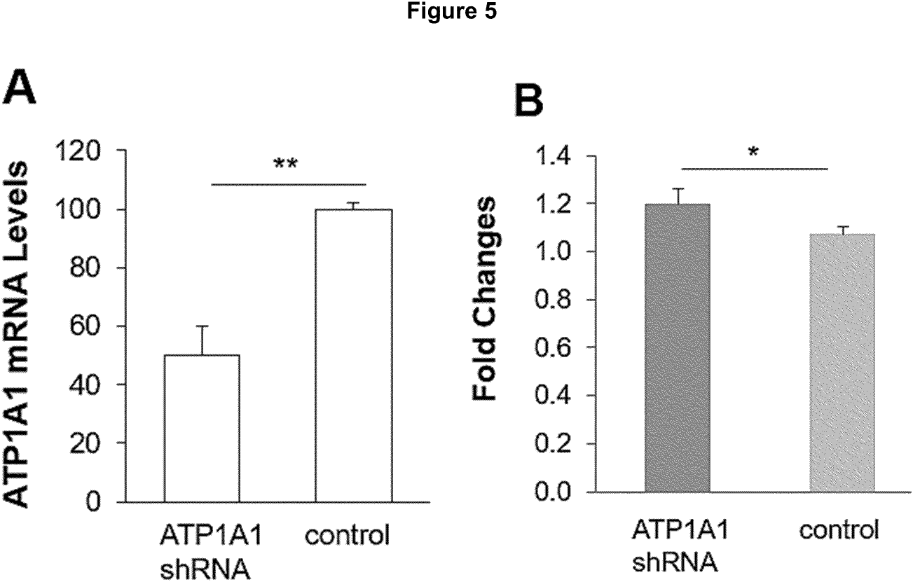

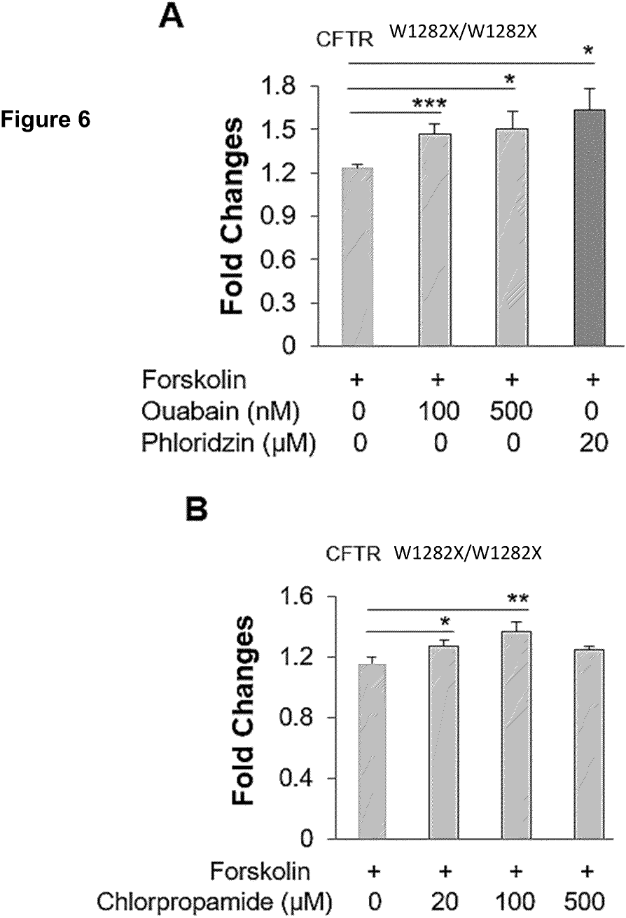

Given that Phlorizin is a SGLT and Na⁺/K⁺-ATPase dual inhibitor (10), we next determined if Na⁺/K⁺-ATPase inhibition also leads to restoration of swelling function of HLOs with Class I mutations. First, we employed ATPA1-specific shRNA to knockdown ATPA1, the a1 subunit in HLOs with Class I mutation (W1282X/W1282X) (FIG. 5A). Selective knockdown of ATPA1 led to a marked increase (approximately 12%) of the swelling size (FIG. 5B), demonstrating Na⁺/K⁺-ATPase is another drug target for therapy of CF with Class I mutations. Consistently, Na⁺/K⁺-ATPase inhibitor Ouabain increased the size of W1282X/W1282X HLOs swelling by 22% compared to 33% increase by Phlorizin (FIG. 6A). We also assessed the effects of a second Na⁺/K⁺-ATPase inhibitor, Chlorpropamide. Chlorpropamide treatment also increased the size of W1282X/W1282X HLOs swelling by 18% (FIG. 6B). Together, these data for the first time demonstrate the therapeutic potential of Na⁺/K⁺-ATPase inhibitors for treatment CF with Class I mutation.

### Dual Inhibition of SGLT1 and Na⁺/K⁺-ATPase Enhances HLO Swelling of Class I CFTR Mutations Than SGLT1 Inhibition Alone

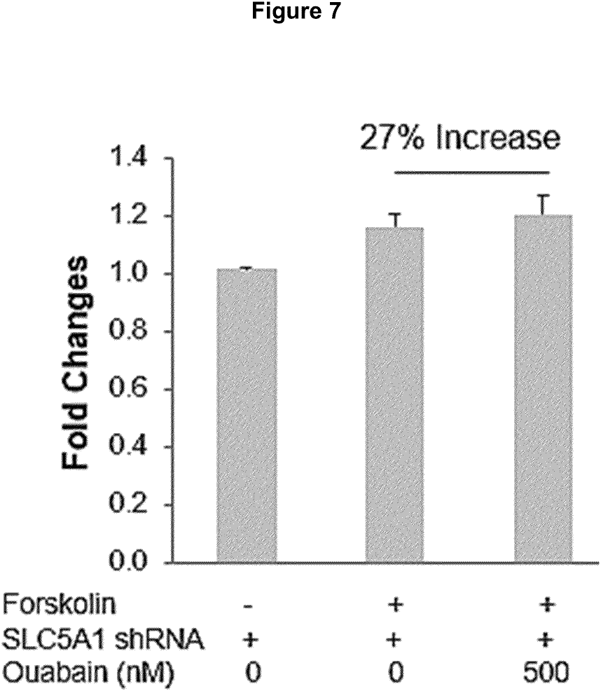

To determine if inhibition of both SGLT1 and Na⁺/K⁺-ATPase will achieve better HLO swelling effects, SGLT1-deficient W1282X/1282X HLOs induced by SGLT1 (i.e. SLC5A1)-specific shRNA treatment were treated with Forskolin and Ouabain or Forskolin and vehicle (no Ouabain), 24h later, quantification of the swelling effects revealed a 27% increase of swelling of Ouabain-treated SGLT 1-deficient HLOs than vehicle-treated SGLT1-deficient HLOs (FIG. 7). These data confirm the superior effects of Phlorizin as a SGLT1 and Na⁺/K⁺-ATPase dual inhibitor.

In summary, our studies have discovered SGLT1 and Na⁺/K⁺-ATPase as drug targets for treatment of CF with Class I mutations. Treatment with the SGLT and Na⁺/K⁺-ATPase dual inhibitor Phlorizin markedly restores the swelling function of HLOs with Class I mutations with 30-40% increase of the swelling size. Thus, SGLT1 inhibitors and Na⁺/K⁺-ATPase inhibitors and more importantly SGLT1 and Na⁺/K⁺-ATPase dual inhibitors such as Phlorizin and its analogs, thereof, have great potential for treatment of Cystic Fibrosis with Class I mutations.

### References

1. Stoltz, D.A., Meyerholz, D.K., and Welsh, M.J. (2015). Origins of cystic fibrosis lung disease. N. Engl. J. Med. 372, 1574-1575.

2. Collins, F.S. (2019). Realizing the dream of molecularly targeted therapies for cystic fibrosis. N. Engl. J. Med. 381, 1863-1865.

3. Riordan, J.R., Rommens, J.M., Kerem, B., Alon, N., Rozmahel, R., Grzelczak, Z., Zielenski, J., Lok, S., Plavsic, N., Chou, J.L., et al. (1989). Identification of the cystic fibrosis gene: cloning and characterization of complementary DNA. Science 245, 1066-1073.

4. Dekkers, J.F., Wiegerinck, C.L., de Jonge, H.R., Bronsveld, I., Janssens, H.M., de Winter-de Groot, K.M., Brandsma, A.M., de Jong, N.W., Bijvelds, M.J., Scholte, B.J., et al. (2013). A functional CFTR assay using primary cystic fibrosis intestinal organoids. Nat. Med. 19, 939-945.

5. McCauley, K.B., Hawkins, F., Serra, M., Thomas, D.C., Jacob, A., and Kotton, D.N. (2017). Efficient derivation of functional human airway epithelium from pluripotent stem cells via temporal regulation of Wnt signaling. Cell Stem Cell 20, 844-857.e6..

6. Sano, R., Shinozaki, Y., and Ohta, T. (2020). Sodium-glucose cotransporters: functional properties and pharmaceutical potential. J. Diabetes Invest. 11, 770-782.

7. Hamosh A, Rosenstein BJ, Cutting GR. (1992). CFTR nonsense mutations G542X and W1282X associated with severe reduction of CFTR mRNA in nasal epithelial cells. Hum Mol Genet. 1, 542-544.

8. Hirai H, Liang X, Sun Y, Zhang Y, Zhang J, Chen YE, Mou H, Zhao Y, Xu J. (2022). The sodium/glucose cotransporters as potential therapeutic targets for CF lung diseases revealed by human lung organoid swelling assay. Mol Ther Methods Clin Dev. 24, 11-19.

9. McCauley, K.B., Hawkins, F., and Kotton, D.N. (2018). Derivation of epithelial-only airway organoids from human pluripotent stem cells. Current protocols in stem cell biology 45, e51.

10. Nakagawa A, and Nakao M. (1977). Localization of the phlorizin site on Na, K-ATPase in red cell membranes. J Biochem. 81, 1511-1515.

### Experimental Procedures

### Definitive Endoderm Cell Differentiation From iPSC

iPSCs were harvested and triturated into single cell suspensions with using Gentle Cell Dissociation Reagent (Stem Cell Technologies, Vancouver, Canada) and seeded onto Corning Matrigel hESC-qualified Matrix (Corning, Corning, NY) coated plate (Corning) in mTesR1 (Stem Cell Technologies) containing 10 µM Y-27632 (Stem Cell Technologies) for 24 hr. Then iPSCs were differentiated into definitive endoderm with using STEMdiff Definitive Endoderm Kit (Stem Cell Technologies) for 72 hr.

### Anterior Foregut Endoderm Cell Differentiation From Definitive Endoderm Cell

Anterior foregut endoderm was differentiated from definitive endoderm cells were treated for 72 hr with anterior foregut endoderm differentiation medium containing Ham’s F-12 Nutrient Mix (Thermo Fisher Scientific) and IMDM (Thermo Fisher Scientific, Waltham, MA) with B27 Supplement (Thermo Fisher Scientific), N2 Supplement (Thermo Fisher Scientific), 0.1% Bovine Serum Albumin Fraction V (Sigma-Aldrich, St. Louis, MO), 1-Thioglycerol (Sigma-Aldrich), 1x GlutaMAX Supplement (Thermo Fisher Scientific), and 1% penicillin-streptomycin, 50 µg/ml L-ascorbic acid, 10 mM SB431542 (Cayman Chemical, Ann Arbor, MI) and 2 mM Dorsomorphin (Cayman Chemical).

### Lung Epithelial Progenitor Differentiation From Anterior Foregut Endoderm Cell

Anterior foregut endoderm cells were treated for 8 days with lung epithelial progenitor differentiation medium containing Ham’s F-12 Nutrient Mix and IMDM with B27 Supplement, N2 Supplement, 0.1% Bovine Serum Albumin Fraction V, 1-Thioglycerol, 1x GlutaMAX Supplement, and 1% penicillin-streptomycin, and 10 ng/ml Human Recombinant BMP4 (Stem Cell Technologies), 50 µg/ml L-ascorbic acid, 3 mM CHIR99021 (Cayman Chemical), and 100 nM Retinoic acid (Sigma-Aldrich).

### Proximal Lung Organoid Differentiation From Lung Epithelial Progenitor

On day 14-15 lung epithelial progenitors were dissociated into single cell suspensions with Trypsin-EDTA (0.05%) (Thermo Fisher Scientific). Harvested cells are immunostained with CD47 and CD26 antibodies. CD47⁺high/positive and CD26⁻ low/negative cells (CD47hi/CD261o) were sorted, followed by resuspending as single cells in 50 µl three-dimensional growth factor reduced Matrigel drops, treated with proximal lung organoid differentiation medium containing Ham’s F-12 Nutrient Mix and IMDM with B27 Supplement, N2 Supplement, 0.1% Bovine Serum Albumin Fraction V, 1-Thioglycerol, 1x GlutaMAX Supplement, and 1% penicillin-streptomycin, 100 ng/ml Human Recombinant FGF10, 250 ng/ml Human Recombinant bFGF (Stem Cell Technologies), 50 µg/ml L-ascorbic acid, 100 nM Dexamethasone (Cayman Chemical), 0.1 mM 8-bromo-Cyclic AMP (8-bromo-cAMP) (Cayman Chemical), and 10 mM 3-isobutyl-1-methylxanthine (Sigma-Aldrich) and Y-27632 (Cayman Chemical).

### Measurement of Forskolin-Induced Swelling of Organoids

CFTR function was quantified by measuring fsk-induced swelling of organoids as described previously (8, 9). Organoids were incubated with or without 10 µM fsk (Selleck Chemicals), and swelling was monitored using time-lapse microscopy. To evaluate the effects of different compounds on the swelling, Amiloride (Selleck Chemicals), Phlorizin (Selleck Chemicals), Empagliflozin (Selleck Chemicals), Ouabain (Selleck Chemicals) or Chlorpropamide (Selleck Chemicals) was added to the culture medium according to the experimental design 24 h before fsk treatment.

### Immunofluorescence Staining

Cells were fixed with 4% Paraformaldehyde (PFA) in PBS for 10 min and permeabilized with 0.5% Triton X-100 (Sigma-Aldrich) in PBS for 5 min at room temperature. Cells were stained with the primary antibody p63 (1:100, CM163A, Biocare Medical) or PE-conjugated human EPCAM (1:200, 12-9326-42, Thermo Fisher Scientific) for 1 hr and secondary antibodies for 45 min at room temperature. Nuclei were counterstained with DAPI.

### shRNA Lentivirus

Lentivirus encoding SGLT-1 shRNA was prepared by transfecting SGLT-1 shRNA Plasmid (h) (Santa Cruz Biotechnology, Inc.), psPAX2 (Addgene), and pCMV-VSV-G (Addgene) into Lenti-X 293T Cell Line with Lipofectamine 3000 (Thermo Fisher Scientific). Lentivirus encoding alpha 1 Sodium Potassium ATPase/ATP1A1 shRNA Plasmid (h) (Santa Cruz Biotechnology, Inc.) was prepared in the same way. shRNA-lentivirus were transduced with 10 µg/ml polybrene into lung epithelial progenitors (8).

### Quantitative RT-PCR

Total RNA was prepared with RNeasy mini kit (Qiagen). cDNA prepared with reverse transcriptase (Applied Biosystems) was applied for qPCR with SYBR Green-based quantitative real-time PCR analysis (Roche Applied Science) was performed with the 7500 fast Real-Time PCR System (Thermo Fisher Scientific) using the following primers. SLC5A1: 5′- CTAAAGCTGATGCCCATGTTC-3′ (SEQ ID NO: 1) and 5′-AGGTTGGATAGGCGATGTTG-3′(SEQ ID NO: 2). ATP1A1: 5′-TGAGATAGTGTTTGCCAGGAC-3′ (SEQ ID NO: 3) and 5′-CAACCCCAATGTCTGCTTTC-3′ (SEQ ID NO: 4). 18srRNA: 5′-CTCAACACGGGAAACCTCAC-3′ (SEQ ID NO: 5) and5′-CGCTCCACCAACTAAGAACG-3′ (SEQ ID NO: 6) was used for the normalization of the levels of mRNA.

### Statistical Analysis

Data are presented as mean ± SEM. Student’s t-test (2-tailed) was used to compare data using GraphPad Prism 8 software (GraphPad Software, Inc., San Diego, CA). P values<0.05 were considered statistically significant.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating cystic fibrosis (CF) characterized by a class I nonsense mutation in the cystic fibrosis transmembrane conductance regulator (CFTR) gene in a subject in need thereof, the method comprising administering to the subject a pharmaceutical composition comprising an effective amount of an inhibitor selected from the group consisting of a sodium glucose co-transporter (SGLT) inhibitor, a Na⁺/K⁺-ATPase inhibitor, an SGLT1/Na⁺/K⁺-ATPase dual inhibitor, and combinations thereof.

2. The method of claim 1, wherein the inhibitor is the SGLT1/Na⁺/K⁺-ATPase dual inhibitor.

3. The method of claim 2, wherein the SGLT⅟ Na⁺/K⁺-ATPase dual inhibitor is phlorizin or analogs thereof.

4. The method of claim 1, wherein the inhibitor is a SGLT inhibitor.

5. The method of claim 4, wherein the inhibitor is a SGLT1-selective inhibitor.

6. The method of claim 4, wherein the inhibitor is a non-selective SGLT inhibitor.

7. The method of claim 4, wherein the SGLT inhibitor is selected from the group consisting of sotagliflozin, phloretin, licogliflozin, SGLT inhibitor 1, mizagliflozin, KGA-2727, SGL5213, LX2761, T-1095, and analogs thereof.

8. The method of claim 1, wherein the inhibitor is a Na⁺/K⁺-ATPase inhibitor.

9. The method of claim 8, wherein the Na⁺/K⁺-ATPase inhibitor is selected from the group consisting of ouabain, bufalin, istaroxime, biacetyl monoxime, rostafuroxin, gitoxin, oleandrin, deslanoside, chloropropamide, periplocin, and analogs thereof.

10. The method of claim 8, wherein the Na⁺/K⁺-ATPase inhibitor is ouabain or chlorpropamide.

11. The method of claim 1, wherein the pharmaceutical composition comprises the SGLT inhibitor and the Na⁺/K⁺-ATPase inhibitor.

12. The method of claim 1 further comprising administering an effective amount of a therapeutic agent selected from the group consisting of a CFTR modulator, a CFTR amplifier, and combinations thereof.

13. The method of claim 12, wherein the inhibitor is the SGLT1/Na⁺/K⁺-ATPase dual inhibitor.

14. The method of claim 12, wherein the inhibitor is the SGLT inhibitor.

15. The method of claim 12, wherein the inhibitor is the Na⁺/K⁺-ATPase inhibitor.

16. The method of claim 12, wherein the therapeutic agent is a CFTR modulator selected from the group consisting of Trikafta, Symdeko, Kalydeco, Orkambi, and combinations thereof.

17. The method of claim 12, wherein the therapeutic agent is Trikafta.

18. The method of claim 12, wherein the inhibitor is phlorizin and the therapeutic agent is Trikafta.

19. The method of claim 1, wherein the class I nonsense mutation is a G542X mutation.

20. The method of claim 1, wherein the class I nonsense mutation is a W1282X mutation.


SEQUENCE LISTING (6 sequences)

SEQ ID NO: 1 - DNA, 21 bp, synthetic construct
  source 1..21: mol_type = other DNA
ctaaagctgatgcccatgttc

SEQ ID NO: 2 - DNA, 20 bp, synthetic construct
  source 1..20: mol_type = other DNA
aggttggataggcgatgttg

SEQ ID NO: 3 - DNA, 21 bp, synthetic construct
  source 1..21: mol_type = other DNA
tgagatagtgtttgccaggac

SEQ ID NO: 4 - DNA, 20 bp, synthetic construct
  source 1..20: mol_type = other DNA
caaccccaatgtctgctttc

SEQ ID NO: 5 - DNA, 20 bp, synthetic construct
  source 1..20: mol_type = other DNA
ctcaacacgggaaacctcac

SEQ ID NO: 6 - DNA, 20 bp, synthetic construct
  source 1..20: mol_type = other DNA
cgctccaccaactaagaacg